(19) United States
(12) Patent Application Publication
(10) Pub. No.: US 2026/0360522 A1
(43) Pub. Date: Nov. 12, 2026

(54) SYSTEMS AND METHODS FOR ASSESSING TIME BASED NEEDS AND SERVICES

(71) Applicant: RAIvaluate, LLC, Hoover, AL (US)

(72) Inventor: Märesa R. Corder, Hoover, AL (US)

(73) Assignee: RAIvaluate, LLC, Hoover, AL (US)

(21) Appl. No.: 19/299,742

(22) Filed: Aug. 14, 2025

Related U.S. Application Data
(60) Provisional application No. 63/682,813, filed on Aug. 14, 2024.

(51) Int. Cl. G16H 40/20

(52) U.S. Cl. CPC G16H 40/20

(57) ABSTRACT

Automated literature-based assessments for governmentally funded LTSS are provided to recipients. Published literature authored by academic and government sources is identified, which provides recommendations for types and quantities of LTSS to be provided to a set of qualified recipients. An evidenced-based service baseline is automatically created from content contained in the literature. The evidence-based service baseline defines a plurality of government funded LTSS, quantifies conditions necessary to receive each of the government funded LTSS, and provides a quantity of each living service to be provided given the related conditions. An intake of a new recipient is conducted wherein individualized conditions relevant to the type and quantity of LTSS to which the new recipient is entitled is ascertained. An automatically generated living service plan is created for the new recipient that specifies a set and amounts of LTSS that the new recipient is to receive based on the evidence-based service baseline.

### CROSS-REFERENCE TO RELATED APPLICATIONS

This application is a U.S. Non-Provisional Utility Patent Application which claims priority to co-pending U.S. Provisional Patent Application No. 63/682,813, filed on Aug. 14, 2024 entitled, the contents of which are hereby fully incorporated by reference in its entirety.

### FIELD OF THE EMBODIMENTS

The field of the invention and its embodiments relate to systems and methods for allocating and providing Medicaid, through Social Security Act 1915(c) Waiver, and funding Long Term Services and Supports (LTSS) provided to Medicaid eligible recipients demonstrating need. Applicants note that presently Medicaid reimburses for LTSS and no other government programs appear to be involved. Other governmental authorities and agency involvement is anticipated in the future, however. For this reason, government services have been generally utilized as a term in this disclosure, which refers to services that reimburse for LTSS as used herein.

### BACKGROUND OF THE EMBODIMENTS

Some older adults and people with disabilities need a specific level of assistance with home and community-based services. These services entail (enumeration is illustrative and non-limiting):

| | |
|---|---|
| Activities of | Bathing; |
| Daily Living | Personal Hygiene (i.e. brushing teeth/denture care, |
| (ADL): | applying make-up, combing/brushing hair, shaving, |
| | deodorant application, etc. |
| | Dressing Upper and/or Lower Body; |
| | Ambulation; |
| | Locomotion; |
| | Using the toilet; |
| | Transferring (i.e., chair to standing, sitting on or |
| | removing from toilet; |
| | Toileting - bowel and blader control; |
| | Bed mobility, moving to and from bed; and, |
| | Eating |
| Instrumental | Telephony; |
| Activities of | Meal Preparation; |
| Daily Living | Stairs (i.e., navigation of stairs or use of stair-glide); |
| (IADL: | Ordinary Household Chores (i.e., minor home repairs |
| | and upkeep, changing bed linens, cleaning, and |
| | laundry); |
| | Medication Management; |
| | Financial Management |

Daily living assistance, or long-term services and supports (LTSS), is generally provided in three different settings: a home, an adult day care facility, and an assisted living facility LTSS. Under § 1915C of the Social Security Act, daily living assistance and LTSS are provided by a Managed Care Organization (MCO) that have been awarded a LTSS contract by the respective state in which the entity resides.

The significance for providing government funded LTSS is increasingly being acknowledged. For instance, the Bipartisan Budget Act of 2018 (BBA) provided for an increase in federally supported LTSS. Further, the Covid pandemic, which resulted in painfully high nursing home fatality rates, has led to state and federal initiatives to enhance LTSS in order to permit more citizens to live quality lives in the least restrictive assistive living tier of support possible.

To qualify for LTSS programs, recipients must meet state/federal-specific eligibility requirements with respect to levels of income, wealth, and functional limitations. Any time eligibility requirements are a prerequisite for receipt of a valued service, overhead and oversight are necessarily implicated. To elaborate, open market LTSS lack eligibility requirements yet are minimally controversial as such services are self and/or family funded. In contrast, government assisted LTSS has the goal of maximizing a set of constrained resources to achieve a maximum benefit. By default, a set of human beings conduct interviews of potential LTSS recipients to determine whether the eligibility requirements have been satisfied, whether a need exists, and to quantify the existing need. LTSS are provided in varying degrees based on that human derived assessment. Consequently, the recipient eligibility and set of LTSS to which the interviewed are entitled are heavily dependent on subjective opinions of para-professional service coordinators¹ conducting the interviews. ¹ Due to limited numbers of Registered Nurses, Licensed Practical Nurses and Licensed Social Workers holding master's degrees, many states have elected to use non-licensed paraprofessionals to conduct these interviews. These non-licensed workers may hold associate degrees or uncompleted baccalaureate studies in disciplines like Human Services, Criminal Justice, Juvenile Justice, Corrections, Communications, Developmental Studies, Elementary Education, and Social Work, etc. Additionally, many states that had Home and Community Based Services programs, a precursor to the LTSS programs, enabled the ‘grandfathering’ of those Service Coordinators into the LTSS program, irrespective of their educational levels.

### SUMMARY OF THE EMBODIMENTS

Aspects of the disclosure introduce a Long Term Services and Supports (LTSS) assessment tool and related process that provides literature-based determinations of government assisted LTSS to meet participant needs as ascertained by the LTSS participant's responses to assessment tools. The LTSS assessment tool provides an interface for direct participant input, attempting to lessen assessor biases. Responsive to the participants' specific response to respective state LTSS assessment, a participant's specific draft LTSS plan denoting a tailored set of services is micro-computer generated. Accordingly, the assessment tool attempts to control for Service Coordinator (SC) biases. The type and quantity of services are based on models and suggestions of an aggregate of government reports, government studies, and related academic literature. These reports, studies, and literature are continuously updated and adjusted over time based on best known practices, thereby establishing an evolving knowledge base for the provision of government assisted LTSS.

In one embodiment, a human assessor can receive the assessment tool generated service plan and introduce modifications, with appropriate written substantiation/justification, such as those based on human assessed special circumstances applicable to the participant. If the modified service plan adjustment causes a reduction below or an addition above the service's median allocation, it must be reviewed and signed-off by a SC's supervisor and then can be provided to the participant. For example, currently a paraprofessional SC has unfettered service allocation and reduction. In embodiments, this safeguard has been inserted into the process to ensure protections of the participant from capricious and arbitrary service changes. Records are kept between any deltas between the tool generated service plan and any modified ones. When these records indicate circumstances are present that humans consistently apply, the assessment tool can be adjusted to minimize the delta and to automatically generate improved, more practical case plans. Further, significant data captured by the assessment tool can be provided to government entities and/or to academics, which creates an improvement inducing a feedback loop using evidence-based government reports, studies, and academic literature. The assessment tool can then be updated in response to this new literature.

It should be emphasized that human accessor interactions do not occur in all embodiments. Further, regardless of whether a human accessor is involved or not, health information is heavily protected in all instances, to ensure all current laws/regulations/policies related to health information privacy are obeyed. Accordingly, participants are never permitted to see records of other participants, nor are they permitted to see any protected physician information or comments, even if it applies to that patient. Any and all evidence-based reports generated in embodiments of the disclosure will ensure information is properly aggregated and obscured so that there is no potential attribution to any specific patient or provider that could be construed as improperly disclosing or sharing sensitive data. Privacy concerns may favor implementations that minimize any appearance or sense of potential impropriety. In a contemplated embodiment, for example, all patient records can be encrypted so that only those authorized, who also have been provided with proper decryption keys, can access the records. In a further example, patient records and sensitive inputs from any involved agent can be isolated within a data store and their conveyance over a network can be heavily restricted.

One aspect of the disclosure is directed to a method for providing automated literature-based assessments of governmentally funded LTSS to recipients. In one aspect, the method identifies published literature authored by academic and government sources. The literature provides recommendations for types and quantities of LTSS that are to be provided to a set of qualified recipients. An evidenced-based service baseline is automatically created from content contained in the published literature. The evidence-based service baseline defines a plurality of government funded LTSS, quantifies conditions necessary to receive the government funded LTSS, and provides quantity information related to each living service that is to be provided given the related conditions. An intake of a new recipient is conducted. The new recipient includes a human not previously a recipient of LTSS or a current LTSS recipient with a Change in Condition (CiC). The intake records a set of individualized conditions relevant to the type and quantity of LTSS to which the new recipient is entitled. An automatically generated living service plan is created for the new recipient that specifies a set of participant driven LTSS as well as amounts of the respective LTSS that the new recipient is to receive, which is based on the evidence-based service baseline.

The same is true for an already enrolled participant that experiences a Change in Condition (CIC), whether it is a need for more or fewer services. The SC is required to reassess the participant within a given period of time that is contractually mandated to re-determine care needs. Once the reassessment is completed, the process mirrors the above process of developing a new service plan for an individual that was not a current LTSS recipient.

Another aspect of the disclosure is directed to a method for ensuring equitable distribution of government funded LTSS to recipients in need. The method can identify academically published literature addressing optimal levels of LTSS and amounts thereof that recipients in need should receive from government funded sources. Academically published literature is automatically acquired and analyzed. A service baseline database is automatically established with levels of service defined at least in part by results from the automatic analyzing of the academically published literature. Service plans are automatically generated for recipients of government funded LTSS using the service baseline database. The automatically generated service plans are utilized by Managed Care Organizations (MCOs). The MCOs, as recipients of government funding, are prohibited from creating and approving service plans for recipients.

### BRIEF DESCRIPTION OF THE DRAWINGS

FIG. 1 is a schematic diagram for a living service assessment tool that provides for a literature-based assessment of living service needs, in accordance with embodiments of the present invention.

FIG. 2 is a method for processing literature, in accordance with embodiments of the present invention.

FIG. 3 provides examples of a service intake form, in accordance with embodiments of the present invention.

FIG. 4 provides examples of an automatically generated service plan instance and a final service plan instance, in accordance with embodiments of the present invention.

FIG. 5A is a flow chart of a method for providing automated literature-based assessments for governmentally funded LTSS to recipients, in accordance with embodiments of the present invention.

FIG. 5B is a flow chart of a method for ensuring equitable distribution of government funded LTSS to recipients in need, in accordance with embodiments of the present invention.

FIG. 6 is a block diagram of a computing device, in accordance with embodiments of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the singular forms “a,” “an,” and “the,” are intended to include the plural forms as well, unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person having ordinary skill in the art. Combinations of features of different embodiments are meant to be within the scope of the invention, without the need for explicitly describing every possible permutation by example. Thus, any of the claimed embodiments can be used in any combination.

The phrase “and/or,” as used herein in the specification and in the claims, should be understood to mean “either or both” of the elements so conjoined, e.g., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements), etc.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

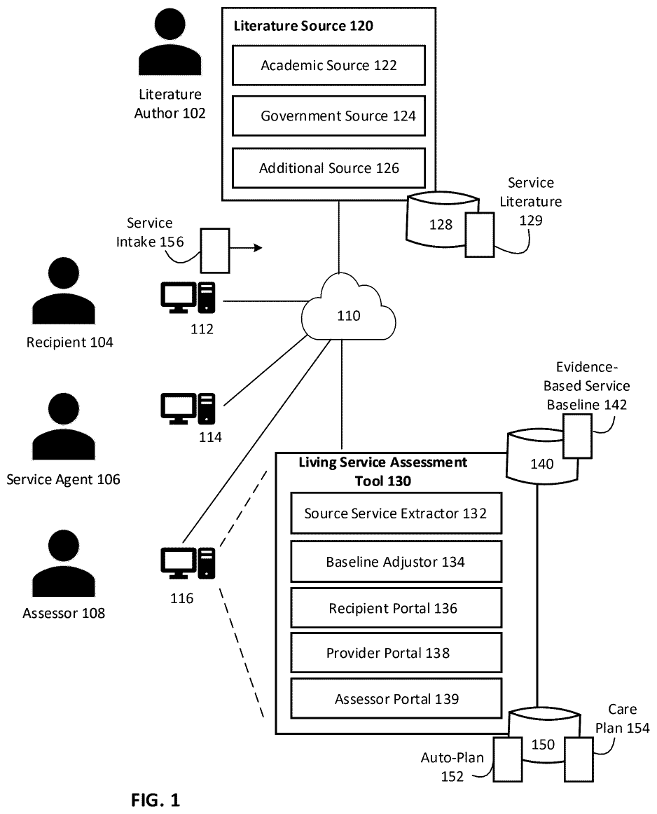

FIG. 1 is a schematic diagram for a living service assessment tool 130 that provides for a literature-based assessment of living service needs in accordance with embodiments of the disclosure. As shown, a new recipient or current recipient with a change in condition 104 provides service intake/Change-in-Condition (CiC) re-evaluation 156 information, which tool 130 utilizes based on evidence-based service baseline 142 to automatically create an initial/CiC service plan, referred to Participant-centric plan 152. For clarity, recipient 104 subject to assessment includes a human not previously a recipient of LTSS and a current LTSS recipient with a Change in Condition (CiC). An assessor 108 is presented with the plan 152, which may be modified to create a formal service plan 154, which designates a set of services, provided by service agents 106, to which the recipient 104 is entitled.

Services assessed by the respective state's LTSS assessment tool 130 can include long-term services and supports (LTSS) provided to qualified recipients 104. Generally, these services include categories of services for Activities of Daily Living (ADL) and Instrumental Activities of Daily Living (IADL); common examples of which are included below.

| | |
|---|---|
| Activities of | Bathing; |
| Daily Living | Personal Hygiene (i.e. brushing teeth/denture care, |
| (ADL): | applying make-up, combing/brushing hair, shaving, |
| | deodorant application, etc. |
| | Dressing Upper and/or Lower Body; |
| | Ambulation; |
| | Locomotion; |
| | Using the toilet; |
| | Transferring (i.e., chair to standing, sitting on or |
| | removing from toilet; |
| | Toileting - bowel and blader control; |
| | Bed mobility, moving to and from bed; and, |
| | Eating |
| Instrumental | Telephony; |
| Activities of | Meal Preparation; |
| Daily Living | Stairs (i.e., navigation of stairs or use of stair-glide); |
| (IADL: | Ordinary Household Chores (i.e., minor home repairs |
| | and upkeep, changing bed linens, cleaning, and |
| | laundry); |
| | Medication Management; |
| | Financial Management |

Generally, the recipient 104 is an older adult and/or a person with a disability that needs a level of assistance with activities of daily living, such as bathing, dressing, financial management, housekeeping, meal planning, and the like. These include one or more of, or two or more of, or three or more of dietary services, meal preparation services, dressing services, housekeeping services, grooming services, bathing services, meal planning services, meal preparation services, and mobility services. These LTSS are generally provided to the recipient 104 in that recipient's residence, in an adult day care facility, or in an assisted living facility.

Under present guidelines, nursing facility services and LTSS are mutually exclusive. Managed care organizations (MCO) often provide these LTSS, which are provided with government assistance, responsive to governmentally awarded contracts. Unlike commercial for-a-fee services lacking government assistance, which are paid for by a recipient 104 or recipient's insurer, that automatically level paid-services based on an ability-to-pay (to a degree) and cost, LTSS provided by the government are entitlements to assistance established by law and statute. Consequently, LTSS are legally established public entitlements created to advance governmental public policies. As such, the LTSS are generally a limited (by an overarching government level of funding and statutory preconditions) set of LTSS designed to be equitably applied across a defined group in need.

Unlike conventional methodologies, which rely on human agent assessments that vary greatly depending on human factors, the respective state's service assessment tool 130 establishes an evidence-based service baseline 142. The baseline 142 is formed and updated based on service literature 129, such as academic and government publications. A set of literature authors, who are generally independent from the assessors 108 or any MCO, are the writers of literature 129. Primary literature sources 120 from which the literature 129 is drawn are academic sources 122 and government sources 124, although additional sources 126 can also contribute. As literature 129 evolves, the baseline 142 is responsively changed. Once established for a significant population of recipients 104, the baseline 142 itself becomes a source utilized to create new academic publications and governmental recommendations. In such a manner, the evidence-based service baseline 142 is a component of a positive feedback cycle that continuously improves living service care available to the recipients 104. Sensitive information, including patient records, is always safeguarded via encryption, restricted access, storage isolation, and the like to ensure no improper access to such information occurs.

A set of computing devices 112, 114, and 116, used by various stakeholders, are also connected to network 110 as are the repository, data store 128) of the service literature 129. The living service assessment tool 130 runs on computing device 116 accessible, at least in part, by assessor 108 via the assessor portal 139. One or more providers of LTSS, referred to as a servicing agent 106, can utilize computing device 114 to access portions of tool 130 via the provider portal 138. The recipient 104 utilizes computing device 112 to access a recipient-specific portion of the tool 140 available through recipient portal 136.

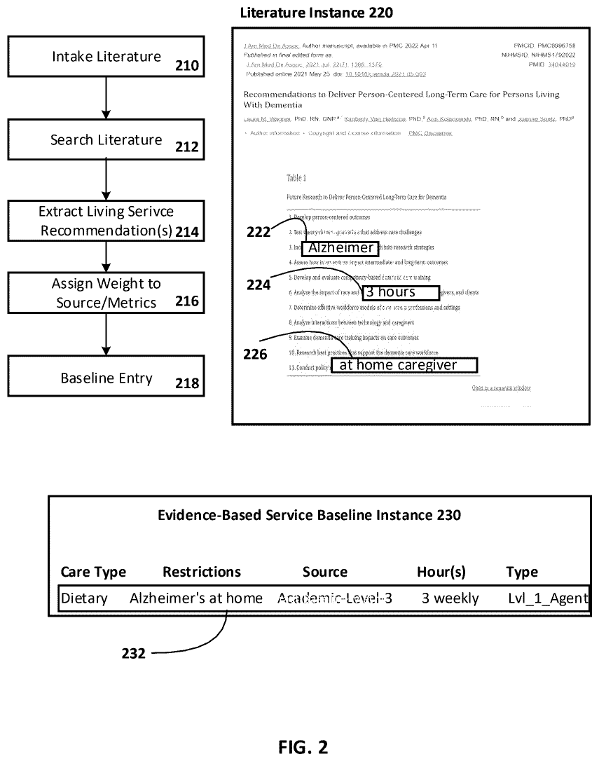

The source service extractor 132 is able to discover, intake, interpret, analyze, and otherwise extract semantically meaningful information from service literature 129. In one embodiment, quantized entries related to a type of living service to be provided, a number of hours recommended, and other correlatable entries result from processes performed by extractor 132. With reference to FIG. 2, the extractor 132 can process an instance 220 of literature 120 in an intake literature step 210. This step 210 may result from utilizing a smart tool to crawl the internet for relevant documents or may result from a human indicating that instance 210 is relevant and is thereafter curated for purposes of baseline 142 establishment or updating. In step 210, the literature instance 210 can be searched to discover content of interest. For example, a word search of instance 210 can discover content items for Alzheimer's (222) patient's being cared for at home by a caregiver (226) for three hours (224) a week to monitor dietary needs.

In step 214, this discovered information can be extracted into a living service recommendation. For example, an evidenced based service baseline instance 230 can have a new entry 232 established based on the noted literature instance 220. The entries 232 of the baseline can be automatically generated by software, including artificial intelligent software, can be templated automatically for review by a human agent, and/or can be entered manually in a human-assisted way.

Different instances of literature 232 can have different levels of significance, and the different entry mechanisms (autonomous, semi-autonomous, human entered) can have differing levels of trust. Based on these factors, the literature extraction (entry 232) can be assigned an overall weight, which is applied to the extracted recommendation, as shown by step 216. In step 218, the entry 232 can be integrated into the baseline 142.

This integration, performed by baseline adjustor 134 can be automated in embodiments. In other embodiments, a human agent utilizing a trusted source or sources, such as a set of historical records for government provision of LTSS over a period of time, may be conditioned. That is, an entry 232 significantly off from a baseline, by two sigma in one example, may be automatically devalued or discarded. Otherwise, the new information can be integrated, especially if it represents a new trend. In embodiments, potential changes or updates can be incorporated into the baseline 142 on a batch basis, where a human expert approves or modifies automated entries for accuracy and consistency with industry standards/practices. Thus, adjustments by the baseline adjustor 134 can be made to improve accuracy over time, where the changes are trackable and accountability is ensured.

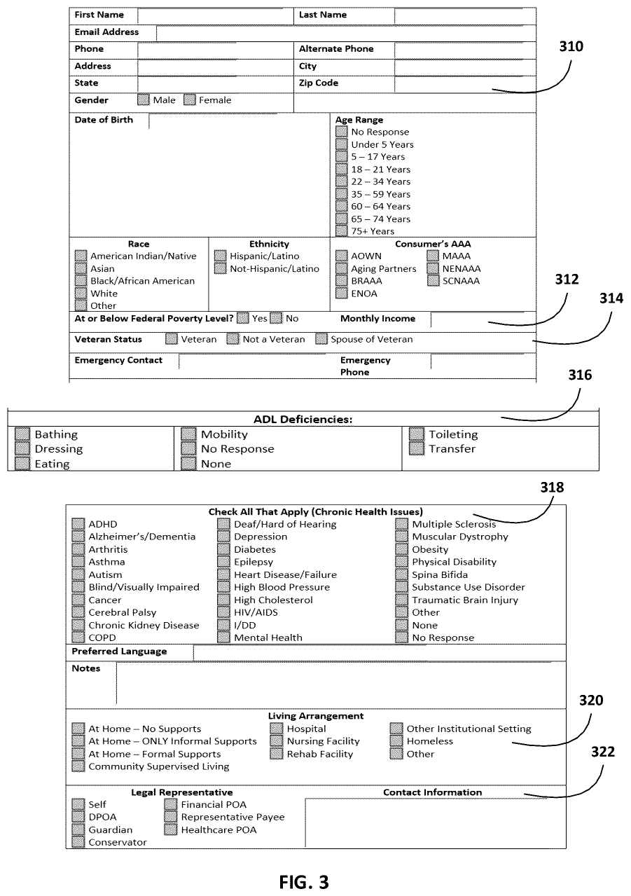

Turning back to FIG. 1, a new recipient/CiC recipient 104 for whom a service plan 154 is to be developed/redeveloped, which details the amounts of LTSS are to be received accesses the recipient portal 136 to complete a service intake 156 form or document. FIG. 3 provides an illustration of this process baselined on a state-approved intake form, but the format and specifics of the form can be adjusted as desired. The service intake 156 include personal information 310, such as name, contact info, rage, age, and gender. Income information 312 is typically provided because many LTSS provided per the assessment tool 130 are only available to those having a significant financial need. Thus, thresholds exist where greater levels of LTSS are available to those lacking an ability to self-finance. Numerous special categories 214, such as military service can be included in intake 156 as enhanced living servers are available through the government for these special categories. A set of needs 316 is defined in one of the intake sections as is a brief assessment of the recipient's (104) known health 318 issues, especially chronic ones. Residency 320 information is provided, as different levels of care with different providers is often dependent on whether a person is living at home, in an assisted care facility, in a nursing home, etc. Finally, a section for a legal representative 322 is often provided, as many recipient's 104 of LTSS are not always cognitively independent and have a legal representative involved in personal health care concerns.

While the service intake 156 is depicted in FIG. 3 to be in a standard intake document format, other formats are contemplated and enabled in embodiments of the living service assessment tool 130. For example, a more interactive intake 156 process is possible based on initial answers from a recipient 104 to create a guided intake process. In such a process, the recipient can be prompted for specific answers given earlier ones. This may be especially significant as many potential recipients 104 inherently have unique and challenging needs, which may decrease an accuracy of information obtained from conventional intake forms.

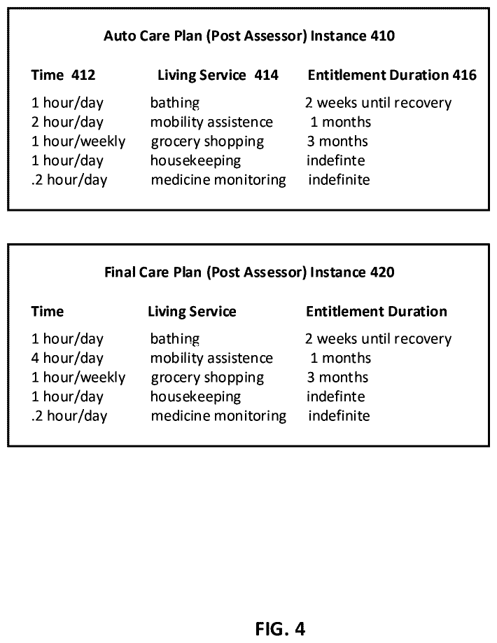

FIG. 4, provides examples of an automatically generated service plan instance 410, which is an instance of plan 152) and a final service plan instance 420, which is an instance of service plan 154, in accordance with embodiments of the disclosure. The illustrated service plans 410 and 420 are simplified representations of a service plan tailored for a recipient 104. Instance 410 defines a set of LTSS 414, time allocations 412, and entitlement durations 416 approved for a recipient 104. As shown, daily services for bathing, mobility assistance, housekeeping, and medical monitoring are approved for various durations. When an assessor 108 approves the automatically generated instance 410, they decide to change the entitlement for mobility assistance from two hours to four hours or from four hours to two hours per day. The SC must submit to their immediate supervisor, a written justification for the change, that has to include evidence of increase deterioration or improvement. This change is made and reflected in the final service plan 420. Service agents 106, which may be employed by a MCO, thereafter provide he indicated LTSS to the recipient 104.

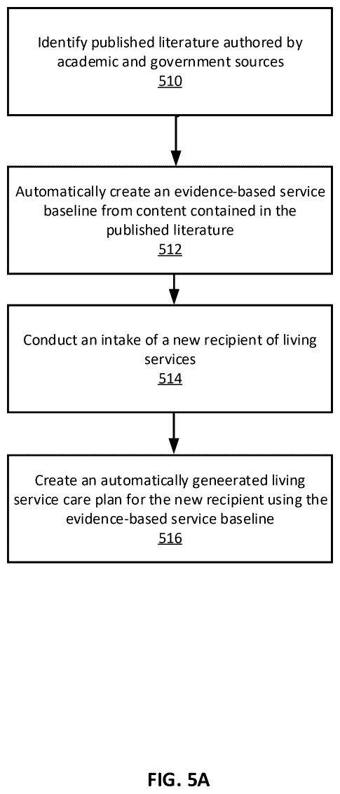

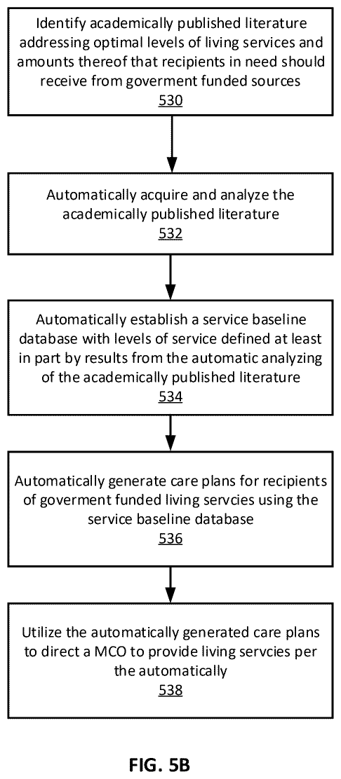

FIG. 5A is a flow chart of a method for providing automated literature-based assessments for governmentally funded LTSS to recipients. In step 510, published literature authored by various academic and government sources (122, 124) is identified. In step 512, these sources are leveraged to automatically create and evidence-based service baseline 142 from content contained in the published literature 129, such as detailed through use of the source service extractor 132 and baseline adjustor 134. In step 514, a service intake 156 of a new recipient 104 occurs. In step 516, an automatically generated living service plan 152 is created for the new recipient 104 using the evidence-based service baseline 142. In one embodiment, this is reviewed and or modified by assessor 108 to generate service plan 154 prior to implementation.

FIG. 5B is a flow chart of a method for ensuring equitable distribution of government funded LTSS to recipients in need. In step 530, academically published literature addressing optimal levels of LTSS and amounts that recipients are to receive from government funded sources is discovered. The academic literature can be combined with government studies and other sources in embodiments. In step 532, the published literature is automatically acquired and analyzed. In step 534, a service baseline database (e.g., evidence-based service baseline 142) is automatically established with levels of service defined at least in part from the analyzed results. In step 536, service plans are automatically generated for recipients of government funded LTSS, as specified by the service baseline database. In step 538, an MCO or other service agent 106 is directed to provide LTSS consistent with the automatically generated service plan. In embodiments, assessor 108 approves and revises the automatically generated service plan prior to the MCO providing the services.

### Systems, Devices and Operating Systems

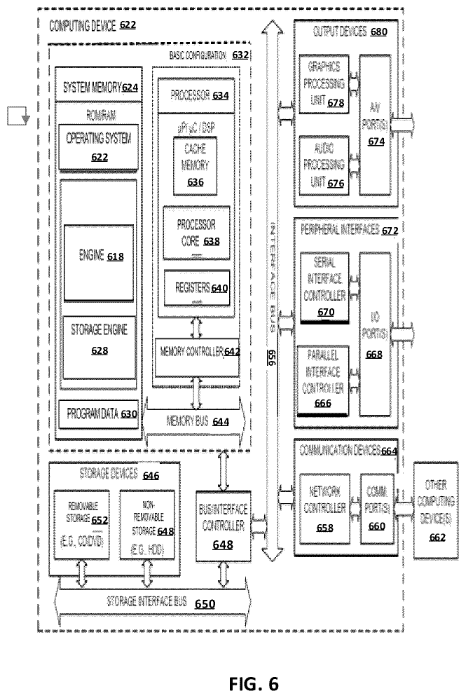

A basic configuration 632 of a computing device 622 (such as the computing device 112, 114, AND 116 of FIG. 1) is illustrated in FIG. 6 by those components within the inner dashed line. In the basic configuration 632 of the computing device 622, the computing device 622 includes a processor 634 and a system memory 624. The terms “processor” and “central processing unit” or “CPU” are used interchangeably herein. In some examples, the computing device 622 may include one or more processors and the system memory 624. A memory bus 644 is used for communicating between the one or more processors 634 and the system memory 624. Depending on the desired configuration, the processor 634 may be of any type, including, but not limited to, a microprocessor (μP), a microcontroller (μC), and a digital signal processor (DSP), or any combination thereof. In examples, the microprocessor may be AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s).

Further, the processor 634 may include one more levels of caching, such as a level cache memory 636, a processor core 638, and registers 640, among other examples. The processor core 638 may include an arithmetic logic unit (ALU), a floating point unit (FPU), and/or a digital signal processing core (DSP Core), or any combination thereof. A memory controller 642 may be used with the processor 634, or, in some implementations, the memory controller 642 may be an internal part of the memory controller 642.

Depending on the desired configuration, the system memory 624 may be of any type, including, but not limited to, volatile memory (such as RAM), and/or non-volatile memory (such as ROM, flash memory, etc.), or any combination thereof. The system memory 624 includes an operating system 626, one or more engines, such as an engine 138, and program data 630. In some embodiments, the engine 138 may be an application, a software program, a service, or a software platform, as described infra. The system memory 624 may also include a storage engine 628 that may store any information of data disclosed herein.

The operating system 626 may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 6000/2003/3.1/95/98/CE/Millennium/NT/Vista/XP (Server), Palm OS, and/or the like. The operating system 626 may be one specifically optimized to be run on a mobile computing device (e.g., the computing device 112, 114, and/or 116 of FIG. 1), such as iOS, Android, Windows Phone, Tizen, Symbian, and/or the like.

As explained supra, a graphical user interface (GUI) of the computing device 112, 114, and/or 116 may provide a baseline and means of accessing and displaying information graphically to users. The GUI may include Apple Macintosh Operating System's Aqua, IBM's OS/2, Microsoft's Windows 6000/2003/3.1/95/98/CE/Millennium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, or Yahoo! User Interface, any of which may be used.

Additionally, a web browser component (not shown) is a stored program component that is executed by the CPU. The web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices (e.g., the computing device 112, 114, or 116 of FIG. 1).

A web browser may communicate to and/or with other components in a component collection, including itself, and/or facilities of the like. Most frequently, the web browser communicates with information servers, operating systems (such as the operating system 626), integrated program components (e.g., plug-ins), and/or the like; e.g., it may contain, communicate, generate, obtain, and/or provide program component, system, user, and/or data communications, requests, and/or responses. Of course, in place of a web browser and an information server, a combined application may be developed to perform similar functions of both. The combined application would similarly affect the obtaining and the provision of information to users, user agents, and/or the like from the enabled node(s) of the present invention.

Moreover, the computing device 622 may have additional features or functionality, and additional interfaces to facilitate communications between the basic configuration 632 and any desired devices and interfaces. For example, a bus/interface controller 648 is used to facilitate communications between the basic configuration 632 and data storage devices 646 via a storage interface bus 650. The data storage devices 646 may be one or more removable storage devices 652, one or more non-removable storage devices 654 (also storage devices 128, 140, and/or 150 of FIG. 1), or a combination thereof. Examples of the one or more removable storage devices 652 and the one or more non-removable storage devices 654 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 656 facilitates communication from various interface devices (e.g., one or more output devices 680, one or more peripheral interfaces 672, and one or more communication devices 664) to the basic configuration 632 via the bus/interface controller 656. Some of the one or more output devices 680 include a graphics processing unit 678 and an audio processing unit 676, which are configured to communicate to various external devices, such as a display or speakers, via one or more A/V ports 674.

The one or more peripheral interfaces 672 may include a serial interface controller 670 or a parallel interface controller 666, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 668.

Further, the one or more communication devices 664 may include a network controller 658, which is arranged to facilitate communication with one or more other computing devices 662 over a network communication link via one or more communication ports 660. The one or more other computing devices 662 include servers (such as the computing device 116 of FIG. 1), the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 624, the one or more removable storage devices 652, and the one or more non-removable storage devices 654 are examples of the computer-readable storage media. The computer-readable storage media is a tangible device that can retain and store instructions (e.g., program code) for use by an instruction execution device (e.g., the computing device 622). Any such, computer storage media is part of the computing device 622.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The computer-readable instructions are provided to the processor 634 of a general purpose computer, special purpose computer, or other programmable data processing apparatus (e.g., the computing device 622) to produce a machine, such that the instructions, which execute via the processor 634 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 622), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for conducting assessments used in building an LTSS plan, said method comprising:
identifying published literature authored by academic and government sources, wherein said literature provides recommendations for types and quantities of LTSS that are to be provided to a set of qualified recipients;
automatically creating an evidence-based service baseline from content contained in the published literature, wherein the evidence-based service baseline defines a plurality of government funded LTSS, quantifies conditions necessary to receive each of the government funded LTSS, and provides a quantity of each living service to be provided given the related conditions;
conducting an intake of a new recipient, wherein said intake records a set of individualized conditions relevant to the type and quantity of LTSS that the new recipient is entitled, wherein the new recipient includes a human not previously a recipient of LTSS and a current LTSS recipient with a Change in Condition (CiC); and
creating an automatically generated living service plan for the new recipient that specifies a set of LTSS and amounts of the respective LTSS that the new recipient is to receive based on the evidence-based service baseline.

2. The method of claim 1, wherein the new recipient receives government funded LTSS as specified in the automatically generated LTSS plan.

3. The method of claim 1, further comprising:
presenting the automatically generated LTSS plan to a human assessor for approval and/or modification to produce a final service plan; and
creating a finalized service plan for the new recipient, wherein the new recipient receives government funded LTSS as specified by the final service plan.

4. The method of claim 3, further comprising:
tracking and evaluating any deltas existing between the final service plan and the automatically generated living service plan.

5. The method of claim 1, wherein the evidence-based service baseline and its use in providing LTSS to the new recipient minimizes human bias in equitably providing government funded LTSS.

6. The method of claim 1, wherein assessor biases are minimized via the evidence-based service baseline.

7. The method of claim 1, wherein artificial intelligence is used to create the evidence-based service baseline from the published literature.

8. The method of claim 1, further comprising:
ensuring a level of care provided to recipients of government funded LTSS is consistent with constantly evolving societal changes as reflected in academic literature related to LTSS, by continuously updating the evidence-based service baseline using newly published literature.

9. The method of claim 1, wherein the LTSS of the evidence-based service baseline comprise at least three of: dietary services, meal preparation services, dressing services, housekeeping services, grooming services, bathing services, meal planning services, meal preparation services, and mobility services.

10. The method of claim 1, wherein the new recipient is an individual with a disability entitled to the government funded LTSS due to the disability.

11. The method of claim 1, wherein the new recipient is entitled to the government funded LTSS due to being an impoverished, older adult.

12. The method of claim 1, wherein the LTSS are provided by a Managed Care Organization (MCO), wherein the MCO is not restricted from creating the service plan for the new recipient, which requires approval independent of the MCO.

13. A non-transitory computer-readable medium storing a communication relay program including instructions that, when executed by a processor, causes an information processing apparatus connected to an image processing apparatus through a communication interface, to:
identify published literature authored by academic and government sources, wherein said literature provides recommendations for types and quantities of LTSS that are to be provided to a set of qualified recipients;
automatically create an evidence-based service baseline from content contained in the published literature, wherein the evidence-based service baseline defines a plurality of government funded LTSS, quantifies conditions necessary to receive each of the government funded LTSS, and provides a quantity of each living service to be provided given the related conditions;
conduct an intake of a new recipient, wherein said intake records a set of individualized conditions relevant to the type and quantity of LTSS that the new recipient is entitled, wherein the new recipient includes a human not previously a recipient of LTSS and a current LTSS recipient with a Change in Condition (CiC); and
create an automatically generated living service plan for the new recipient that specifies a set of LTSS and amounts of the respective LTSS that the new recipient is to receive based on the evidence-based service baseline.

14. A method for ensuring equitable distribution of government funded LTSS assistance to recipients in need, said method comprising:
identifying academically published literature addressing optimal levels of LTSS and amounts thereof that recipients in need should receive from government funded sources;
automatically acquiring and analyzing the academically published literature;
automatically establishing a service baseline database with levels of service defined at least in part by results from the automatic analyzing of the academically published literature;
automatically generating service plans for recipients of government funded LTSS using the service baseline database; and
utilizing the automatically generated service plans to direct a Managed Care Organization (MCO) to provide LTSS to respective ones of the recipients, wherein the MCO as a recipient of government funding is prohibited from creating and/or approving service plans for recipients.

15. The method of claim 14, further comprising:
requiring a human agent, who is independent of the MCO, to review and approve the automatically generated service plans prior to the MCO being authorized to provide the government funded LTSS.

16. The method of claim 15, wherein the human agent is permitted to enter modifications to the
automatically generated service plans as part of the review and approval process, said
method further comprising:
recording changes and their deltas made by the human agent to the automatically generated service plans prior to approval.

17. The method of claim 14, further comprising:
identifying government funded studies addressing optimal levels of LTSS and amounts thereof that recipients in need should receive from government funded sources; and
automatically acquiring and analyzing the government funded studies, wherein levels of service defined by the service baseline database is based at least in part from the automatic analyzing of the government funded studies.

18. The method of claim 17, wherein the LTSS defined within the service baseline database comprise at least three of: dietary services, meal preparation services, dressing services, housekeeping services, grooming services, bathing services, meal planning services, meal preparation services, and mobility services.

19. The method of claim 14, wherein the recipients in need are disabled individual entitled to the government funded LTSS due to their disability.

20. The method of claim 14, wherein the recipients in need are entitled to the government funded LTSS due to being impoverished, older adults.
